(19) United States
(12) Patent Application Publication
(10) Pub. No.: US 2010/0222785 A1
(43) Pub. Date: Sep. 2, 2010

(54) ANCHOR DRIVER

(75) Inventors: John E. Fitzpatrick, Brighton, MA (US); Jason E. Mitchell, Greenbrier, TN (US); Robert F. Labadie, Nashville, TN (US); J. M. Fitzpatrick, Nashville, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Vanderbilt University, Nashville, TN (US)

(21) Appl. No.: 12/700,935

(22) Filed: Feb. 5, 2010

Related U.S. Application Data
(63) Continuation-in-part of application No. 12/185,434, filed on Aug. 4, 2008.
(60) Provisional application No. 60/963,295, filed on Aug. 3, 2007.

(51) Int. Cl. A61B 17/00

(52) U.S. Cl. 606/104

(57) ABSTRACT

A surgical instrument for securing an anchor in a target area of the skull of a living subject to a predetermined driving depth. In one embodiment, the surgical instrument has a driver member, a bit member, a cap member that has a body portion forming a bore therein along a longitudinal axis, wherein inside the bore, a first inner surface is formed with an engagement length and a shape that is complimentary to a shaped surface of a driving engagement portion of the driver member to allow it to have a slidable engagement with the cap member, and a base member that has a body portion and an anchor engagement portion, wherein the engagement length of the first inner surface of the cap member equals the predetermined driving depth.

### CROSS-REFERENCE TO RELATED PATENT APPLICATION

This application is a continuation-in-part of U.S. patent application Ser. No. 12/185,434 filed on Aug. 4, 2008, entitled “Anchor Driver With Assured Seating” by J. Michael Fitzpatrick, Robert F. Labadie and Jason E. Mitchell, which itself claims benefit pursuant to 35 U.S.C. §119(e), of U.S. provisional Application Ser. No. 60/963,295, filed Aug. 3, 2007 entitled “Anchor Driver with Assured Seating” by J. Michael Fitzpatrick, Robert Labadie, and Jason E. Mitchell, the disclosure for which is hereby incorporated herein in its entirety by reference.

Some references, if any, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### STATEMENT OF FEDERALLY-SPONSORED RESEARCH

The present invention was made with Government support awarded by the National Institute on Deafness and Other Communication Disorders under contract No. 1R01DC008408-01A. The United States Government has certain rights to this invention pursuant to this grant.

### FIELD OF THE INVENTION

The present invention generally relates to a surgical instrument, and more particularly relates to a surgical instrument for securing an anchor in a target area of the skull of a patient.

### BACKGROUND OF THE INVENTION

Image-guided surgery technology has been clinically available since the mid-1980s. Analogous to global positioning systems (GPS), image-guided surgery facilitates intraoperative surgical navigation by linking preoperative radiographs to intraoperative anatomy. Central to the image-guided surgery process is registration: the linking of the radiographic images to the patient. To achieve high accuracy, the registration is based on fiducial markers that are identified both in the radiographs and on the patient. Fiducial markers need to be positioned by anchors, which have been used in other medical procedures as well.

A current system for image-guided surgery requires that self-tapping anchors be screwed into a patient's skull through an incision in the scalp. The fact that the anchor cannot be seen by a medical professional such as a surgeon during the screwing because of overlying scalp makes it difficult for the surgeon to know when and whether the anchor is fully seated against the skull. The anchor is placed into a driver that is inserted through the incision. The surgeon presses on the driver and begins to twist. If the surgeon twists too far, the threads that are tapped into the skull by the anchor will be stripped. If the surgeon does not twist far enough, the threads on the anchor will not be fully buried in the skull. In either case, the anchor will not be secure.

Therefore, a heretofore unaddressed need still exists in the art to address the aforementioned deficiencies and inadequacies.

### SUMMARY OF THE INVENTION

Practicing the present invention may create an ideal situation where the base of the anchor is seated against the skull of a patient, at which point the anchor threads are fully buried and the threads in the skull are not stripped. This invention makes it possible for the surgeon to know when and whether this point it reached, such that proper seating is assured. In other words, in one aspect, the present invention provides for placement of the threaded component with a pre-specified penetration into the bone surface of the patient.

The term “patient” as used herein encompasses, in addition to a living human subject, a living animal subject or cadaver, a human cadaver, or other living subjects.

The present invention, in one aspect, relates to a surgical instrument for securing an anchor in a target area of the skull of a patient, where the anchor has a tip portion and a base portion, and wherein the tip portion is configured to penetrate into the targeted area and with a first length l₁ equal to a predetermined driving depth, and the base portion configured to support the tip portion at a first end and has an engagement portion formed at an opposite, second end.

In one embodiment, the surgical instrument has a driver having a first end, an opposite, second end, and a body portion defined therebetween and along a longitudinal axis, wherein a driving engagement portion and a protruding portion protruding away from the driving engagement portion are formed at the second end along the longitudinal axis of the driver, and wherein the driving engagement portion is formed with a second length l₂ along the longitudinal axis and a shaped surface.

The surgical instrument also has a bit that has a base portion with a first end, an opposite, second end, and a shaped surface formed therebetween, and a driving engagement portion formed with an engagement tip and protruding away from the base portion at the second end of the base portion along a longitudinal axis of the bit, wherein a bore is formed proximate to the first end and with a shape that is complimentary to the shape of the protruding portion of the driver to allow the protruding portion of the driver to be received therein.

Furthermore, the surgical instrument has a cap that has a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with a third length l₃ along the longitudinal axis and a shape that is complimentary to the shaped surface of the driving engagement portion of the driver to allow the shaped surface of the driving engagement portion to be received therein.

Moreover, the surgical instrument has a base that has a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein.

The shaped surface of the driving engagement portion of the driver is cross-sectionally hexagonal, cylindrical, oval or polygonal. The shape of the first inner surface of the cap is cross-sectionally hexagonal, cylindrical, oval or polygonal. The shape of the second inner surface of the cap is formed with a shape that is complimentary to the shape of the body portion of the base to allow the body portion to be received therein.

In one embodiment, the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

In one embodiment, the driving engagement portion of the driver is formed with a third diameter d₃, and the protruding portion of the driver is formed with a fourth diameter d₄, and wherein the third diameter d₃ and the fourth diameter d₄ satisfy the relationship of d₃>d₄, such that a step portion is formed at the intersection of the driving engagement portion and the protruding portion of the driver.

In one embodiment, the surgical instrument further includes fastening means for fastening the base and the cap together once the body portion of the base is received in the bore. The fastening means can be one or more screws, one or more mechanical fastening members such as nuts and bolts combinations, and the like.

In one embodiment, the surgical instrument further has a resilient member configured to be received in the bore of the body portion of the base and in contact with the second end of the base portion of the bit and the step portion of the base for providing a tension force to the bit and the base, respectively. The resilient member can be a spring, a metal plate, and the like.

In one embodiment, the engagement tip of the driving engagement portion is formed with a shape that is complimentary to the shape of the engagement portion of the anchor.

In one embodiment, the second length l₂, and the third length l₃ satisfy the relationship of l₂≧l₃.

In operation, the bit is received in the bore of the body portion of the base with its engagement tip engaging the engagement portion of the anchor, the combination of the bit and the base is received in the bore of the cap through the second end, and the driving engagement portion with the protruding portion of the driver is received in the bore of the cap through the first end with a slidable engagement with the first inner surface of the bore of the cap, respectively, such that as the driver is twisted, the driving engagement portion and the protruding portion of the driver engage with the bit and apply a pressure force to the bit, which in turn rotates the anchor into the target area to a depth that equals the first length l₁ at which the driving engagement portion of the driver disengages with the first inner surface of the bore of the cap to allow the driver rotates alone.

In one embodiment, the bit is formed with a fourth length l₄ along the longitudinal axis A from the first end to the end of the driving engagement portion proximate to the engagement tip, and the base is formed with a fifth length l₅ along the longitudinal axis A from the first end to the end of the anchor engagement portion distal from the body portion, respectively.

In one embodiment, the base portion of the anchor is formed with a sixth length l₆ along the longitudinal axis A, and the second length l₂, the fourth length l₄, the fifth length l₅, and the sixth length l₆ satisfy the relationship of l₂+l₄+l₆<l₅.

In one embodiment, the bit member is formed with a hollow well therein. The protruding portion of the driver is formed with a seventh length l₇ along the longitudinal axis A, the hollow well is formed with an axial depth or eighth length l₈ along the longitudinal axis A, and the seventh length l₇ and the eighth length l₈ satisfy the relationship of l₇≧l₈.

The present invention, in another aspect, relates to a surgical instrument for securing an anchor. In one embodiment, the surgical instrument has a driver having a first end, an opposite, second end, and a body portion defined therebetween and along a longitudinal axis, wherein a driving engagement portion and a protruding portion protruding away from the driving engagement portion are formed at the second end along the longitudinal axis of the driver, and wherein the driving engagement portion is formed with a shaped surface.

The surgical instrument further has a bit that has a base portion with a first end, an opposite, second end, and a shaped surface formed therebetween, and a driving engagement portion formed with an engagement tip and protruding away from the base portion at the second end of the base portion along a longitudinal axis of the bit, wherein a bore is formed proximate to the first end and with a shape that is complimentary to the shape of the protruding portion to allow the protruding portion to be received therein.

Moreover, the surgical instrument has a cap that has a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with a shape that is complimentary to the shaped surface of the driving engagement portion to allow the shaped surface of the driving engagement portion to be received therein.

Furthermore, the surgical instrument has a base having a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein.

The shaped surface of the driving engagement portion is cross-sectionally hexagonal, cylindrical, oval or polygonal. The shape of the first inner surface of the cap is cross-sectionally hexagonal, cylindrical, oval or polygonal. The shape of the second inner surface of the cap is formed with a shape that is complimentary to the shape of the body portion of the base to allow the body portion to be received therein.

In one embodiment, the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

In one embodiment, the driving engagement portion of the driver is formed with a third diameter d₃, and the protruding portion of the driver is formed with a fourth diameter d₄, and wherein the third diameter d₃ and the fourth diameter d₄ satisfy the relationship of d₃>d₄, such that a step portion is formed at the intersection of the driving engagement portion and the protruding portion of the driver.

In one embodiment, the engagement tip of the driving engagement portion is formed with a shape that is complimentary to the shape of the engagement portion of the anchor.

In one embodiment, the surgical instrument further includes fastening means for fastening the base and the cap together once the body portion of the base is received in the bore. The fastening means can be one or more screws, one or more mechanical fastening members such as nuts and bolts combinations, and the like.

In one embodiment, the surgical instrument further has a resilient member configured to be received in the bore of the body portion of the base and in contact with the second end of the base portion of the bit and the step portion of the base for providing a tension force to the bit and the base, respectively. The resilient member can be a spring, a metal plate, and the like.

In operation, the bit is received in the bore of the body portion of the base with its engagement tip engaging the engagement portion of the anchor, the combination of the bit and the base is received in the bore of the cap through the second end, and the driving engagement portion with the protruding portion of the driver is received in the bore of the cap through the first end with a slidable engagement with the first inner surface of the bore of the cap, respectively, such that as the driver is twisted, the driving engagement portion and the protruding portion of the driver engage with the bit and apply a pressure force to the bit, which in turn rotates the anchor into the target area until the driving engagement portion of the driver disengages with the first inner surface of the bore of the cap.

The present invention, in a further aspect, relates to a surgical instrument for securing an anchor in a target area of the skull of a living subject to a predetermined driving depth. In one embodiment, the surgical instrument has a driver member having a first end, an opposite, second end, and a body portion defined therebetween and along a longitudinal axis, wherein a driving engagement portion is formed at the second end along the longitudinal axis of the driver, and wherein the driving engagement portion is formed with a shaped surface.

The surgical instrument further has a bit member that has a base portion with a first end, an opposite, second end, and a shaped surface formed therebetween, and a driving engagement portion formed with an engagement tip and protruding away from the base portion at the second end of the base portion along a longitudinal axis of the bit.

Moreover, the surgical instrument has a cap member that has a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with an engagement length and a shape that is complimentary to the shaped surface of the driving engagement portion to allow the driving engagement portion of the driver member to have a slidable engagement with the cap member.

Furthermore, the surgical instrument has a base member that has a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein, wherein the engagement length of the first inner surface of the cap member equals the predetermined driving depth.

The shaped surface of the driving engagement portion is cross-sectionally hexagonal, cylindrical, oval or polygonal. The shape of the first inner surface of the cap is cross-sectionally hexagonal, cylindrical, oval or polygonal. The shape of the second inner surface of the cap is formed with a shape that is complimentary to the shape of the body portion of the base to allow the body portion to be received therein.

In one embodiment, the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

In one embodiment, the surgical instrument further includes fastening means for fastening the base and the cap together once the body portion of the base is received in the bore. The fastening means can be one or more screws, one or more mechanical fastening members such as nuts and bolts combinations, and the like.

In one embodiment, the surgical instrument further has a resilient member configured to be received in the bore of the body portion of the base and in contact with the second end of the base portion of the bit and the step portion of the base for providing a tension force to the bit and the base, respectively. The resilient member can be a spring, a metal plate, and the like.

These and other aspects of the present invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 shows schematically a partial view of a surgical instrument according to one embodiment of the present invention;

FIG. 2 shows schematically a partial view of a surgical instrument according to another embodiment of the present invention;

FIG. 2A shows schematically partial views of the indicator and anchor with a threaded well of the surgical instrument according to the embodiment shown in FIG. 2;

FIG. 3 shows schematically a partial view of a surgical instrument having a driving portion twistably engaged with a powered driving means, according to yet another embodiment of the present invention;

FIG. 3A shows schematically a partial view of a bit and fitting of the surgical instrument according to the embodiment shown in FIG. 3;

FIG. 4 shows schematically a partial view of a surgical instrument according to yet another embodiment of the present invention;

FIG. 5 shows schematically a partial view of a surgical instrument according to yet another embodiment of the present invention;

FIG. 6 shows schematically a partial view of a surgical instrument according to yet another embodiment of the present invention;

FIG. 7 shows schematically a sectional view of a surgical instrument according to one embodiment of the present invention in operation: (a) the surgical instrument engages an anchor or surgical screw and ready to drive it to a target area; (b) the surgical instrument drives the anchor or surgical screw to penetrate into a target area; and (c) the surgical instrument drives the anchor or surgical screw to reach a proper seating in the target area; and

FIG. 8 shows schematically a partial view of a surgical instrument according to the embodiment of the present invention as shown in FIG. 7: (a) an exploding view of the surgical instrument with an anchor or surgical screw; (b) a sectionally exploding view of the surgical instrument with an anchor or surgical screw; and (c) a perspective view of the surgical instrument with the anchor or surgical screw.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise.

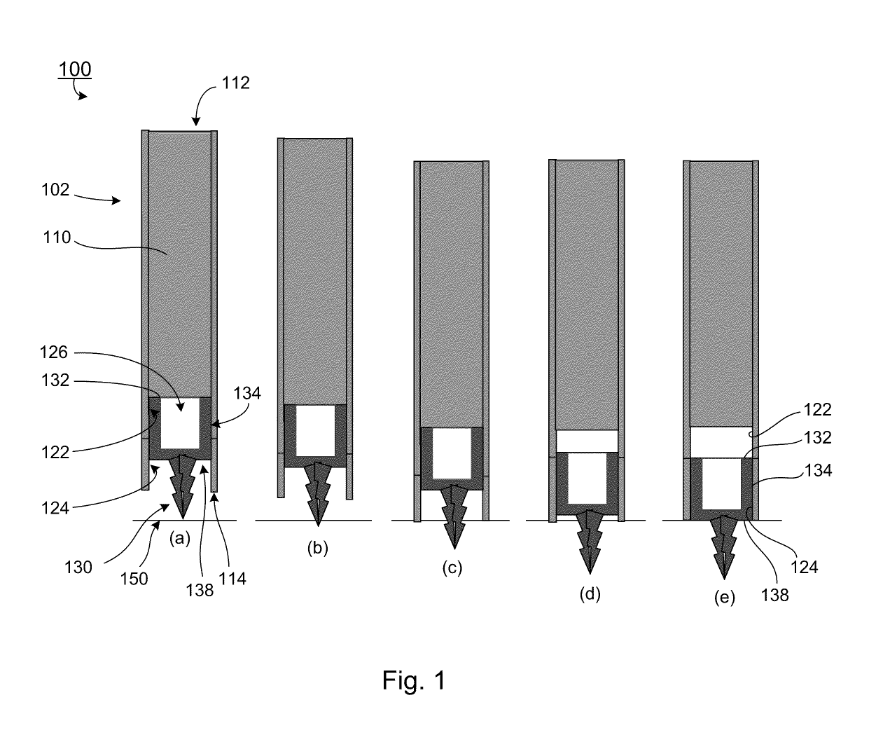

The description will be made as to the embodiments of the present invention in conjunction with the accompanying drawings in FIGS. 1-8.

### Embodiment 1

Referring now to FIG. 1, the present invention, in one aspect, relates to a surgical instrument 100 for securing an anchor 130 in a target area of the skull 150 of a patient, where the anchor 130 has a shaped surface 134, a top 132, and a base 138. In one embodiment of the present invention as shown, the surgical instrument has a driver 102 with a first end 112, an opposite, second end 114, and a body portion 110 defined between the first end 112 and second end 114. The body portion has a bore 126 inside, along a longitudinal axis, where the bore 126 is configured to allow the anchor 130 to be received inside. A first inner surface 122 is formed inside the bore 126 proximate to the first end 112, with a shape that is complimentary to the shaped surface 134 of the anchor 130. A second inner surface 124, having a shape that is different from the shape of the first inner surface 122, is also formed inside the bore 126, between the first inner surface 122 and the second end 114.

In one embodiment, the shaped surface 134 of the anchor 130 is cross-sectionally hexagonal, the first inner surface 122 is cross-sectionally hexagonal, and the second inner surface 124 is cross-sectionally cylindrical. The first inner surface 122 is dimensioned to engage the shaped surface 134 of the anchor 130, and the second inner surface 124 is dimensioned to allow the anchor 130 to slide inside the bore 126. In operation, as shown in FIG. 1(a-e), as the driver 102 is twisted the anchor 130 is driven into the skull 150 to a depth at which the base 138 of the anchor 130 is aligned with the second end 114 of the driver 102 and is seated on the surface of the skull 150, while the top 132 of the anchor is within the second inner surface 124 of the driver 102, as shown in FIG. 1(e). In one embodiment, the depth is adjustable. At least one of the first inner surface 122 and second inner surface 124 is dimensioned to allow the depth to be adjustable.

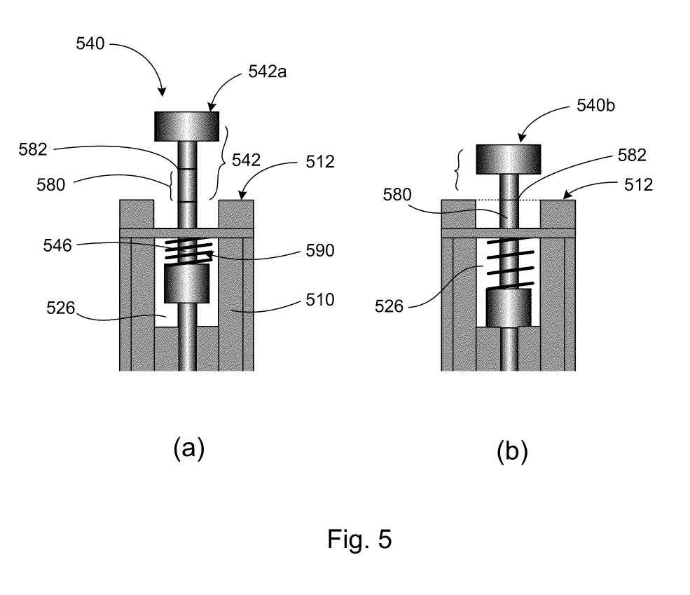

As shown in FIG. 1, the surgical instrument 100 has no moving parts. However, a moving indicator may be added, such as that shown in the embodiment of FIG. 5, or some other means of indicating seating, such as by using electrical or optical components, may be provided. As the driver 102 is twisted, as shown in FIG. 1(a) and FIG. 1(b), the driver 102 exerts downward force on the anchor 130 to push the anchor 130 into the skull 150. It is noted that the anchor 130 does not begin to pull itself into the skull 150 until the driver 102 has made contact with the skull, which is illustrated in FIG. 1(c). The anchor 102 descends (see progression shown in FIG. 1(c-d)) to a point (shown in FIG. 1(e)) at which its base 138 is flush with the second end 114 of the driver 102 and the base 138 is against the skull 150, sitting just below the hexagonal first inner surface 122 of the driver 102. At this point, because it is below the first inner surface 122, it is automatically released from the applied torque, which prevents any danger of stripping threads.

Again referring to FIG. 1, as shown the outer surface 134 of the anchor 130 is hexagonal and the second inner surface 124 is cylindrical. When the anchor 130 is located within this portion of the driver 102, the driver 102 cannot exert torque or force on the anchor 130. The first inner surface 122 is hexagonal such that when the driver 102 is twisted it will exert torque on the anchor 130. The first inner surface 122 does not require an exact hexagonal shape, but it needs to engage the hexagonal outer surface 134 of the anchor 130 in order to exert torque. The driver 102 shown in this exemplary embodiment can be used manually, like a hand-drill, or alternatively it may be driven by a motor that engages the driver at its top or side portions.

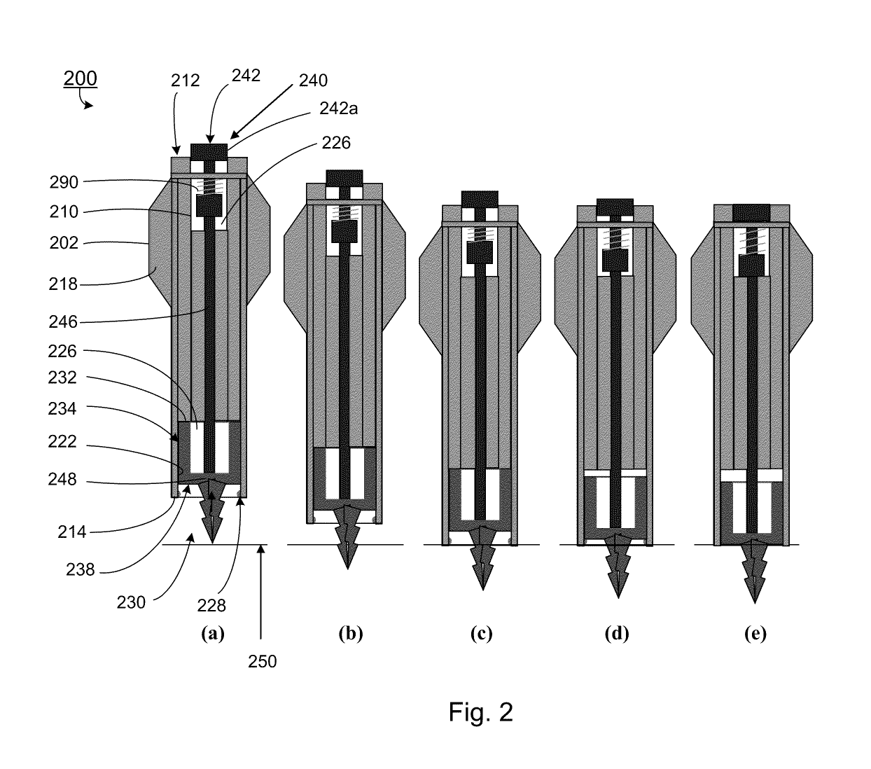

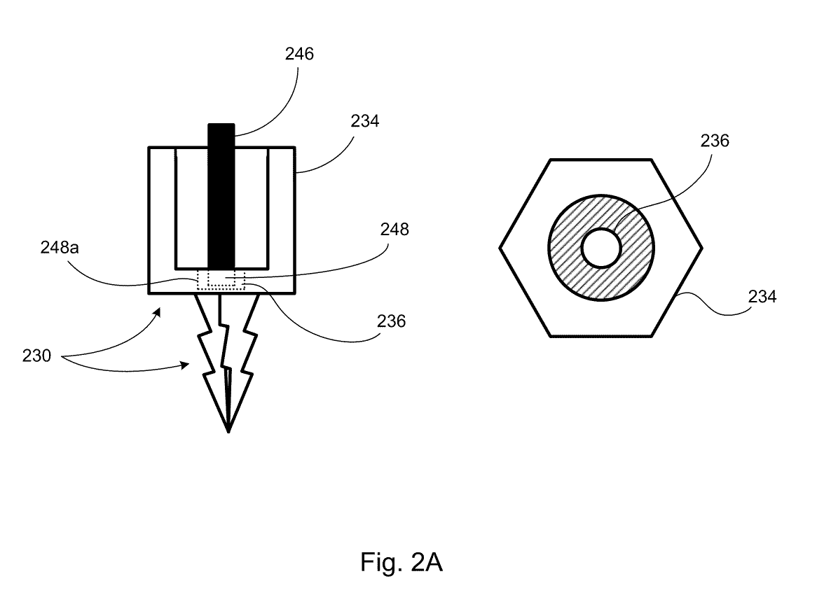

Referring now to FIGS. 2 and 2A, in another aspect, the present invention relates to a surgical instrument 200 for securing an anchor 230 in a target area of the skull 250 of a patient, where the anchor 230 has a shaped surface 234, a top 232, and a base 238. In one embodiment as shown, the surgical instrument 200 has a driver 202 with a first end 212, an opposite second end 214, and a body portion 210 that is defined between the first end 212 and the second end 214, which forms a bore 226 inside along a longitudinal axis. The bore 226 is configured to allow the anchor 230 to be received inside it. A first inner surface 222 is formed inside the bore 226 proximate to the second end 214, and has a shape that is complimentary to the shaped surface 234 of the anchor 230. An indicator 240 is formed to be received in the bore 226 through the first end 212, where the indicator 240 has a body 246, an engaging portion 248 formed in one end of the body 246 for engaging the anchor 230, and an indicating portion 242 formed in the other end of the body 246 for indicating a position of the anchor 230 during operation.

In one embodiment, the engaging portion 248 is formed with a threaded surface 248a that is complimentary to a corresponding threaded well 236 formed within the anchor 230. At least one wing portion 218 is formed on an outer surface of the driver 202. Also, the indicator 240 has a resilient element 290 that is positioned below the indicating portion 242. At least one protrusion 228 is formed on the second inner surface 224 proximate to the second end 214 of the body portion 210, for causing a snap fit with the anchor 230. The indicating portion 242 has a button 242a that extends at least partially above the first end 212 of the driver 202. In operation, as the driver 202 of the surgical instrument 200 is twisted, the anchor 230 is driven into the skull 250 to a depth at which the base 238 of the anchor 230 is aligned with the second end 214 of the driver 202 and is seated on the surface of the skull 250, while the button 242a is inside the first end 212 of the driver 202.

Referring now to FIGS. 2 and 5, FIG. 5 shows another embodiment of the present invention, in which the indicator 540 has an indicating band 580 with a seating indication mark 582 on its body portion 546. In operation, as the button 542a is twisted, the anchor 230 is driven into the skull 250 to a depth at which the base 238 of the anchor 230 is aligned with the second end 214 of the driver 202 and is seated on the surface of the skull 250, while the seating indication mark 582 is aligned with the first end 512 of the driver 202, at (b).

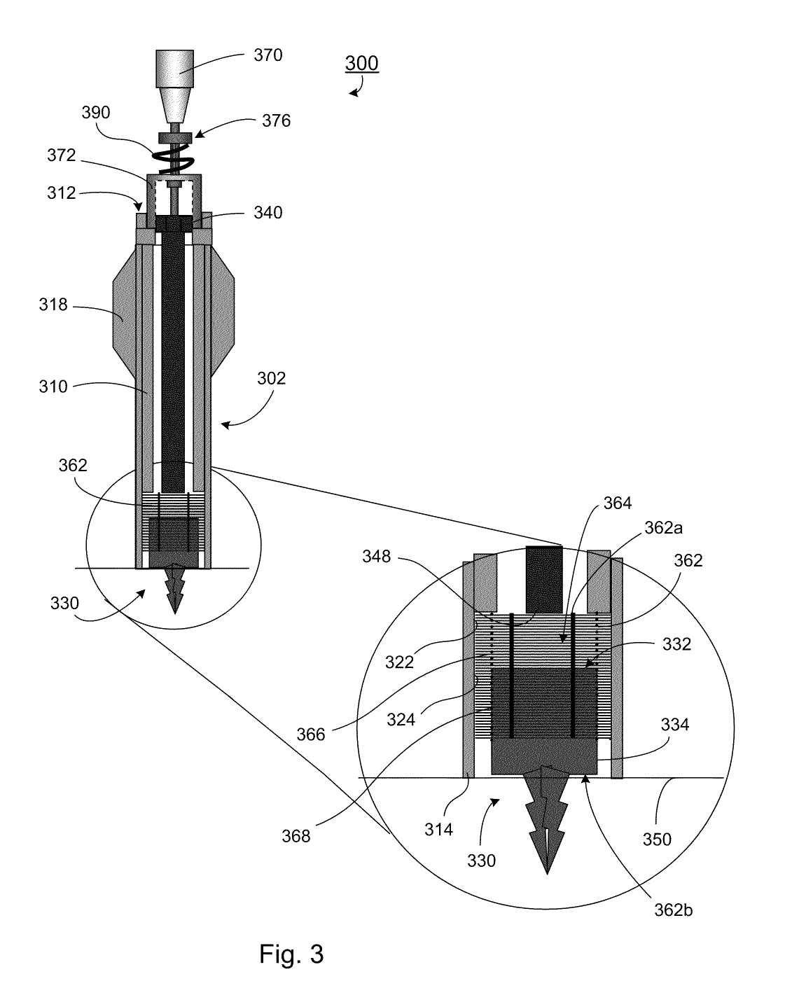

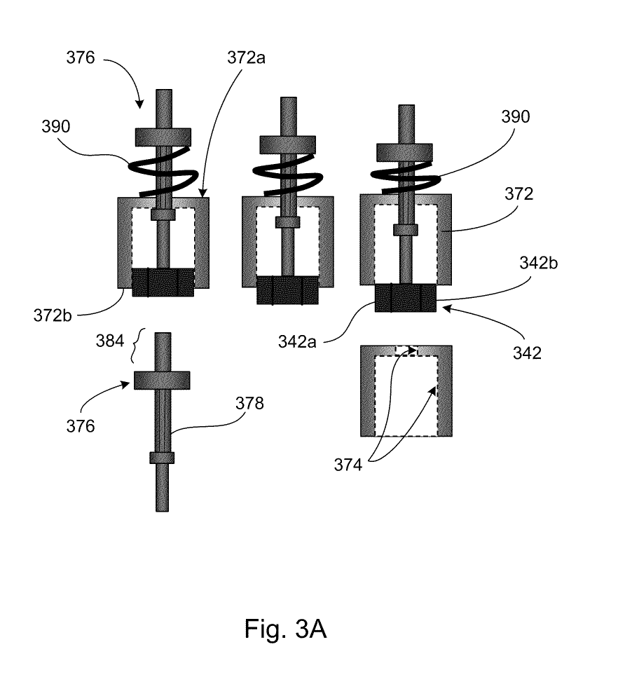

Referring now to FIG. 3, in yet another aspect, the present invention relates to a surgical instrument 300 for securing an anchor 330 in a target area of the skull 350 of a patient, where the anchor 330 has a shaped surface 334. In one embodiment as shown, the surgical instrument has a driver 302 with a first end 312 and an opposite second end 314. A body portion 310 is defined between the first end 312 and the second end 314, and a bore 326 is formed inside the body portion 310 along a longitudinal axis. An indicator 340 is configured to be received in the bore 326 through the first end 312. The indicator 340 has a body portion 346, an engaging portion 348 formed in one end for engaging the anchor 330, and an indicating portion 342 having a button 342a, that is formed in the other end. A receptacle 362 is configured to be received in the bore 326 of the body portion 310 of the driver 302, proximate to the second end 314. The receptacle 362 has a first end 362a, a second end 362b, and an inner cavity 364. A first inner surface 366 is formed inside the inner cavity 364 of the receptacle 362, proximate to the first end 362a of the receptacle 362. The first inner surface 366 has a shape that is complimentary to the shaped surface 334 of the anchor 330. A second inner surface 368, which has a shape that is different from that of the first inner surface 366 of the receptacle 362, is also formed inside the inner cavity 364 of the receptacle 362, between the first inner surface 366 and the second end 362b.

In one embodiment, the shaped surface 334 of the anchor 330 is cross-sectionally hexagonal, the first inner surface 366 of the receptacle 362 is cross-sectionally hexagonal, and the second inner surface 368 of the receptacle 362 is cross-sectionally cylindrical. The engaging portion 348 of the indicator 340 engages with both the receptacle 362 and the anchor 330, and at least one wing portion 318 is formed on an outer surface of the body portion 310 of the surgical instrument 300.

In one embodiment, the surgical instrument 300 has a bit 376 with a driving portion 384 and a first outer surface 378, where the driving portion 348 is formed to be twistably engaged with a powered driving means 370. A fitting 372 is formed with a dimension to allow the button 342a to be received inside. The fitting 372 has a first end 372a, a second end 372b, and an inner surface 374 formed with a shape that is complimentary to the shape of the outer surface 378 of the bit 376 and the outer surface of the button 342a. The fitting 372 engages with both the bit 376 and the button 342a, respectively. A resilient element 390 is positioned below the driving portion 384 of the bit 376 and above the fitting 372. In operation, as the bit 376 is twisted by the powered driving means 370, the anchor 330 is driven into the skull 250 to a depth at which the base 138 of the anchor 330 is aligned with the second end 314 of the driver 302 and is seated on the surface of the skull 250, the top 332 of the anchor 330 is within the first inner surface 366 of the receptacle 362, and the button 342 is below the second end 372b of the fitting 372.

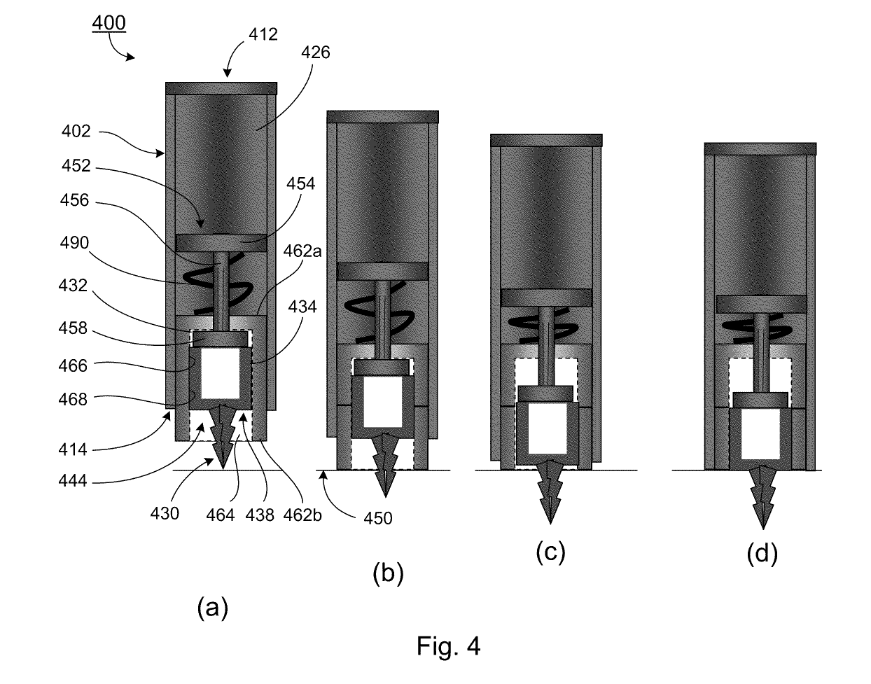

Referring now to FIG. 4, in yet another aspect, the present invention relates to a surgical instrument 400 for securing an anchor 430 in a target area of the skull 450 of a patient, where the anchor 430 has a shaped surface 434, a top 432, and a base 438. In one embodiment as shown, the surgical instrument has a driver 402 with a first end 412, an opposite second end 414, and a body portion 410 that is defined between the first end 412 and the second end 414. A bore 426 is formed in the body portion 410 along a longitudinal axis. A receptacle 462, having a first end 462a, a second end 462b, and an inner cavity 464, is configured to be received at least in part of the bore 426, proximate to the second end 414 of the body portion 410. A first inner surface 466 is formed inside the inner cavity 464 of the receptacle 462 and proximate to its first end 462a, and is formed with a shape that is complimentary to the shaped surface 434 of the anchor 430. A second inner surface 424 is formed inside the inner cavity 434 of the receptacle 462, between the first inner surface 466 and the second end 462b of the receptacle 462, and is formed with a shape that is different from the shape of the first inner surface 466. The surgical instrument 400 also has a driving mechanism 452 for engaging the receptacle 462 to cause it to move in operation.

In one embodiment, the shaped surface 434 of the anchor 430 is cross-sectionally hexagonal, the first inner surface 466 of the receptacle 462 is cross-sectionally hexagonal, and the second inner surface 468 of the receptacle 462 is cross-sectionally cylindrical. Also, at least one wing portion 418 is formed on an outer surface of the driver 402. In operation, as the driver 402 is twisted, the anchor 430 is driven into the target area of the skull 450 to a depth at which the base 438 of the anchor 430 is aligned with the second end 414 of the driver 402 and the second end 462b of the receptacle 462, the base 438 of the anchor 430 is seated on the surface of the skull 450, and the top 432 of the anchor 430 is within the second inner surface 468 of the receptacle 462. The driving mechanism 452 of the surgical instrument comprises a top portion 454, an opposite bottom portion 458, and a body portion 456 formed between the top portion 454 and bottom portion 458. The top portion 454 is formed with a shape that is complimentary to the inner surface 422 of the driver 402, and the body portion 456 of the driving mechanism 452 is formed with a shape that is complimentary to the inner surface 466 of the receptacle 462. The top portion 454 of the driving mechanism 452 engages with the driver 402 and the body portion 456 of the driving mechanism 452 engages with the receptacle 462, respectively. A resilient member 490 is positioned below the top portion 454 of the driving mechanism 452 and below the first end 462a of the receptacle 462. In operation, as a force is applied to the first end 412 of the driver 402 in a direction towards the target area of the skull 450, the driver 402 is pushed against the resistive force of the resilient member 490 until the bottom portion 458 of the driving mechanism 452 engages the top 432 of the anchor 430. As the driver 402 is twisted, the anchor 430 is driven into the target area of the skull 450 to a depth at which the base 438 of the anchor 430 is aligned with the second end 414 of the driver 402 and the second end 462b of the receptacle 462, the base 438 of the anchor 430 is seated on the surface 468 of the skull 450, and the top 432 of the anchor 430 is within the second inner surface 468 of the receptacle 462.

In another embodiment (not shown), a second release mechanism could be implemented, which would activate in the event that pressure is removed from the anchor. This acts as a failsafe measure for a situation where the user is distracted and does not realize that he or she is turning the anchor without applying sufficient pressure to keep it engaged. In this embodiment, if the user of the surgical instrument did not apply enough pressure to engage the anchor, the driver would merely spin without applying torque. The second release mechanism would comprise a second resilient member configured such that the user would have to overcome its opposing resistive force in order to engage the driver. The resilient member would preferably have the same or higher strength than the first resilient member, such that no torque could be applied without applying pressure to it. Alternatively, instead of using a second resilient member, the upper end of the first resilient member could be used for this purpose.

### Embodiment 2

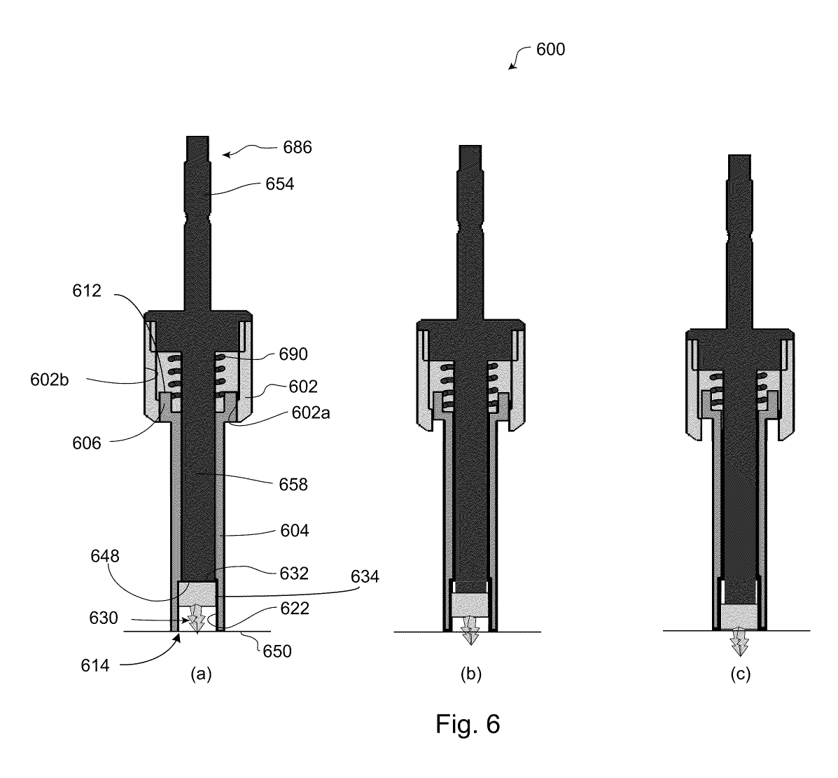

Referring now to FIGS. 6 and 6A, in yet another aspect the present invention relates to a surgical instrument 600 for securing an anchor 630 in a target area of the skull 650 of a patient, where the anchor 630 has a shaped surface. In one embodiment, the surgical instrument 600 has a driver shaft 604, where at one end 614, the driver shaft 604 has a first inner surface 622 which slidably engages with the outer surface 634 of the anchor 630. The cross-sectional shape of the driver shaft 604 prohibits relative rotation between the driver shaft 604 and anchor 630, but allows for relative axial motion. An opposite end 612 of the driver shaft 604 has a top portion 606 of the driver shaft 604, which has an outer surface that slidably engages with the first inner surface 602a of the driver base 602. The shape of the outer surface of the top portion 606 prohibits relative rotation between the driver shaft 604 and driver base 602, but allows for relative axial motion. The height of the top portion 606 of the driver shaft 604 is equivalent to the depth to which the anchor 630 is to be driven. A resilient element 690 is disposed between the driver shaft 604 and driving mechanism 652, which places a force, in a direction towards the anchor 630, on the driver shaft 604 that keeps the driver shaft 604 firmly seated against the skull 650. The driving mechanism 652 has a top portion 686 that is formed to be twistably engaged with a rotationally powered tool, such as a powered driving means as shown in FIG. 3. The body portion 656 of the driving mechanism 652 has threads on its outer surface, external threads which engage the driver base 602. A bottom engaging portion 648 of the driving mechanism 652 engages with and transmits axial force to the anchor 630.

In operation, the engaging portion 686 of the driving mechanism 652 is rotated, such as through the use of a powered driving means (see FIG. 3) that provides a force directed towards the anchor 630, and a rotational force. As the threads of the anchor 630 engage deeper into the skull 650, the anchor 630 moves downward along the vertical axis relative to the driver shaft 604. The driving mechanism 652 moves downward along the vertical axis with the anchor 630 such that, in progression, as shown between steps (a), (b), and (c) of FIG. 6, less and less of the outer surface of the top portion 606 of the driver shaft 604 is engaged, as the anchor 630 is driven further into the skull 650. As shown at step (c), at the point at which the hexagonal portion on the top portion 606 of the driver shaft 604 are no longer in contact and the driver shaft 604 is no longer rotationally engaged with the first inner surface 602a of the driver base 602, the anchor is seated against the skull 650 at the correct depth. As such, the driver shaft 604 is then stationary, as is the anchor 630, although the driving mechanism 652 and driver base 602 may still be rotating.

In one embodiment, the depth to which the anchor 630 is to be driven is adjustable, and at least one of the first inner surface 602a of the driver base 602 and the outer surface portion of the top portion 606 of the driver shaft 604 is dimensioned to allow the depth to be adjustable.

In one embodiment, the engaging surfaces of the driver shaft 604, driver base 602, and driving mechanism 652 are hexagonal. However, these surfaces are not limited as such and could be formed as another geometric shape that allows relative axial motion while transmitting rotational motion. Further, the device of this embodiment is not limited to driving an anchor 630. Any threaded fastener which is itself screwed into the skull 650, or is used as a fastener to hold another component in place, can be used in conjunction with this embodiment of the present invention.

### Embodiment 3

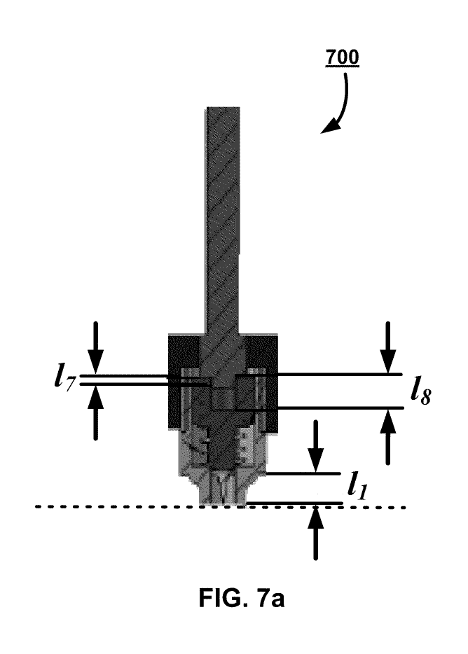

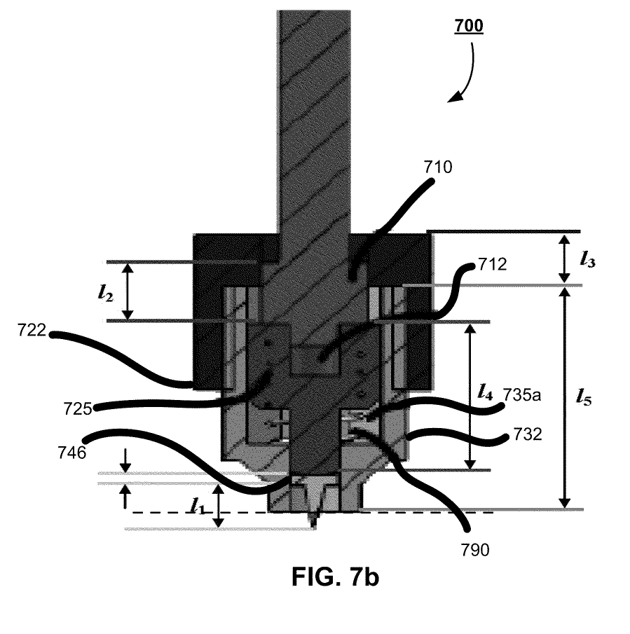

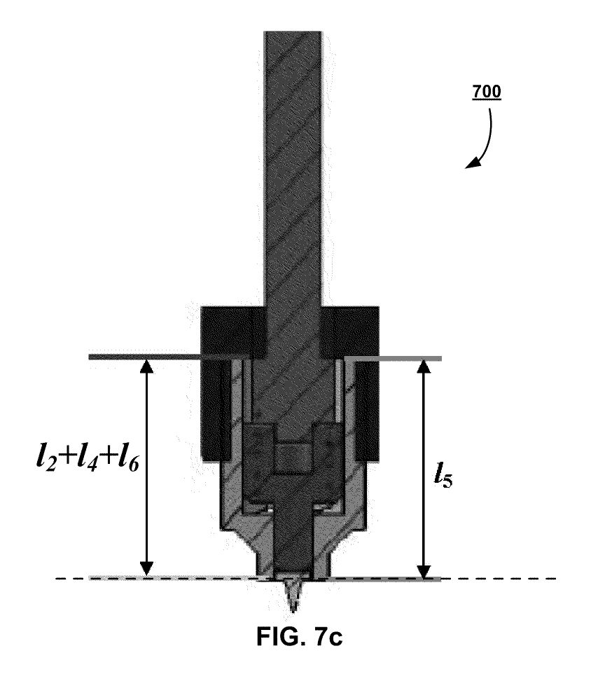

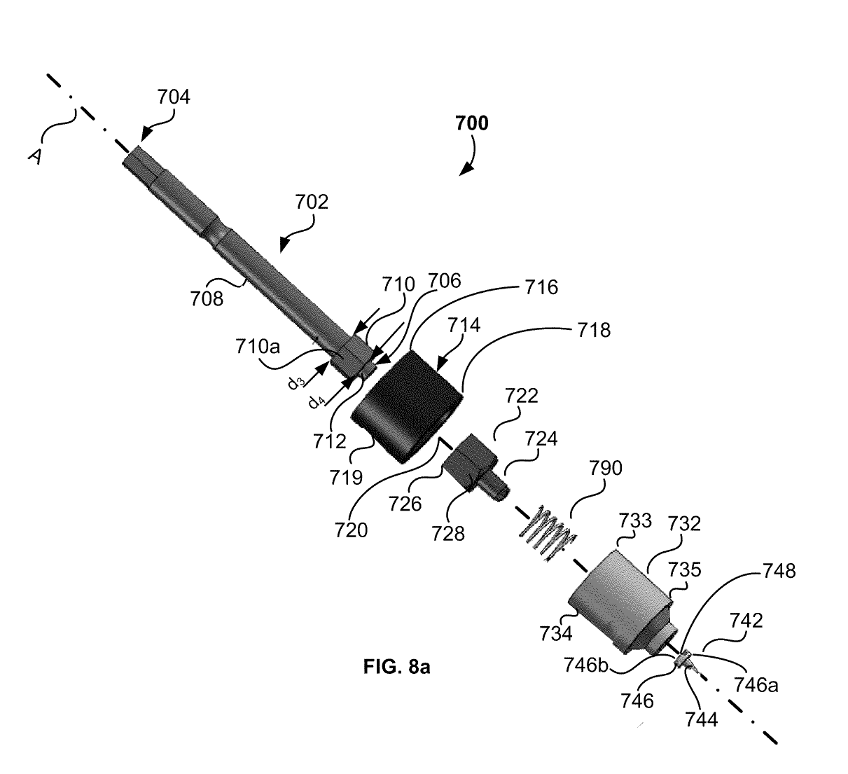

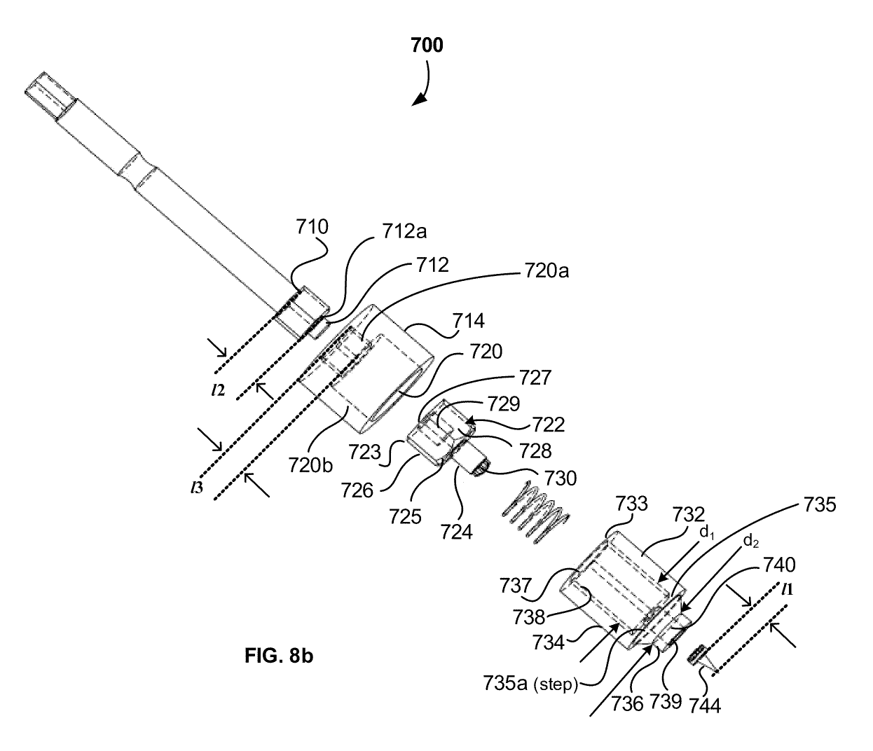

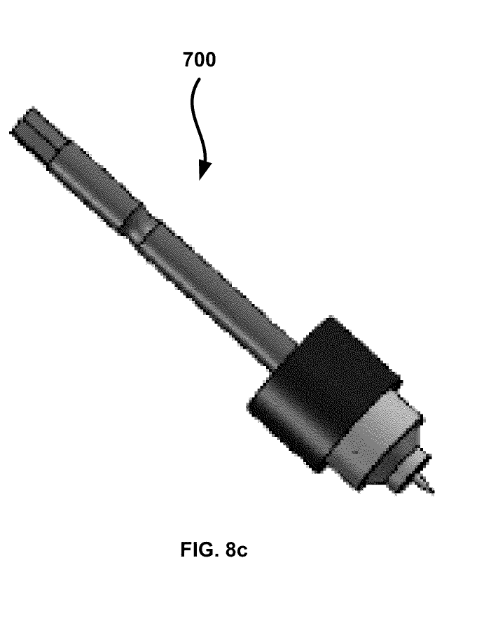

Referring further now to FIGS. 7 and 8, in yet another aspect, the present invention relates to a surgical instrument 700 for securing an anchor 742 in a target area of the skull (not shown but partially represented as a dash line in FIG. 7) of a patient, wherein the anchor 742 has a tip portion 744 and a base portion 746, and wherein the tip portion 744 is configured to penetrate into the targeted area and with a first length l₁ equal to a predetermined driving depth, and the base portion 746 configured to support the tip portion 744 at a first end 746a and has an engagement portion 748 formed at an opposite, second end 746b. The surgical instrument 700 can be used in relation to other types of anchors or surgical screws as well.

In one embodiment as shown in FIGS. 7 and 8, the surgical instrument 700 has a driver or driver member 702 that has a first end 704, an opposite, second end 706, and a body portion 708 defined therebetween and along a longitudinal axis A. A driving engagement portion 710 and a protruding portion 712, which is protruding away from the driving engagement portion 710 along the longitudinal axis A, are formed at the second end 706 along the longitudinal axis A of the driver 702. Moreover, the driving engagement portion 710 is formed with a second length l₂ along the longitudinal axis A and a shaped surface 710a.

Furthermore, the surgical instrument 700 has a cap or cap member 714 that has a first end 716, an opposite, second end 718, and a body portion 719 defined therebetween and forming a bore 720 therein along a longitudinal axis. Inside the bore 720, a first inner surface 720a is formed proximate to the first end 716 and a second inner surface 720b is formed between the first inner surface 716 and the second end 718, respectively, where the first inner surface 720a is formed with a third length l₃ along the longitudinal axis and a shape that is complimentary to the shaped surface 710a of the driving engagement portion 710 to allow the shaped surface 710a of the driving engagement portion 710 of the driver 702 to be received therein. The third length l₃ may also be named as an engagement length of the first inner surface 720a of the cap member 714.

The surgical instrument 700 also has a bit or bit member 722 that has a base portion 726 with a first end 723, an opposite, second end 725, and a shaped surface 728 formed therebetween, and a driving engagement portion 724 formed with an engagement tip 730 and protruding away from the base portion 726 at the second end 725 of the base portion 726 along a longitudinal axis of the bit 722. A bore 727 is formed proximate to the first end 723 and with a shape that is complimentary to the shape of the protruding portion 712 to allow the protruding portion 712 of the driver 702 to be received therein. A hollow well 729 is defined inside the bite 722 along the longitudinal axis of the bit 722 as well. Moreover, a fourth length l₄ along the longitudinal axis A can be defined for the bit or bit member 722 from the first end 723 to the end of the driving engagement portion 724 proximate to the engagement tip 730, as shown in FIG. 7(b).

Additionally, the surgical instrument 700 has a base or base member 732 that has a body portion 734 with a first end 733, an opposite, second end 735, and a bore 737 formed therebetween, and an anchor engagement portion 736 protruding away from the second end 735 of the body portion 734 and with a bore 739 in communication and co-axial with the bore 737 of the body portion 734. Inside the bore 737 of the body portion 734, a first inner surface 738 is formed with a shape that is complimentary to the shaped surface 728 of the bit 722 to allow the bit 722 to be received therein, and inside the bore 739 of the anchor engagement portion 736, a second inner surface 740 is formed with a shape that is complimentary to the shape of the driving engagement portion 724 formed with an engagement tip 730 to allow the driving engagement portion 724 formed with an engagement tip 730 to slidably move therein. It is further noted that a fifth length l₅ along the longitudinal axis A can be defined for the base or base member 732 from the first end 733 to the end of the anchor engagement portion 736 distal from the body portion 734, as also shown in FIG. 7(b).

For the benefits of readers of this disclosure, a sixth length l₆ along the longitudinal axis A can be defined for the base portion 746 of the anchor 742 as shown in FIG. 7(b).

The shaped surface 710a of the driving engagement portion 710 is cross-sectionally hexagonal, cylindrical, oval or polygonal. The shape of the first inner surface 720a of the cap 714 is correspondingly cross-sectionally hexagonal, cylindrical, oval or polygonal. In one embodiment as shown in FIGS. 7 and 8, the shaped surface 710a of the driving engagement portion 710 is cross-sectionally hexagonal, and the shape of the first inner surface 720a of the cap 714 is correspondingly cross-sectionally hexagonal, which are configured such to allow a slidable engagement between the two. Other types of engagement using different geometries such as internal crossed (Phillips), triangle or slotted pattern can also be used to practice the present invention.

The shape of the second inner surface 720b of the cap 714 is formed with a shape that is complimentary to the shape of the body portion 734 of the base 732 to allow the body portion 734 to be received therein.

The surgical instrument 700 may further have fastening means for fastening the base 732 and the cap 714 together once the body portion 734 of the base 732 is received in the bore 720. The fastening means can be one or more fasteners, one or more screws, one or more mechanical fastening members such as nuts and bolts combinations, and the like. Any threaded fastener which is itself a screw, or is used as a fastener to hold another component in place, can be used in conjunction with this embodiment of the present invention.

Now still referring to FIGS. 7 and 8, the first inner surface 738 of the bore 737 in connection with the base 732 is formed with a first diameter d₁, and the second inner surface 740 of the bore 737 in connection with the base 732 is formed with a second diameter d₂, and as formed, the first diameter d₁ and the second diameter d₂ satisfy the relationship of d₁>d₂, such that a step portion 735a is formed at the intersection of the first inner surface 738 and the second inner surface 740 of the bore 737.

The surgical instrument further has a resilient member 790 that is configured to be received in the bore 737 of the body portion 734 of the base 732 and in contact with the second end 725 of the base portion 726 of the bit 722 and the step portion 735a of the base 732 for providing a tension force to the bit 722 and the base 732, respectively. The resilient member can be a spring, a metal plate, and the like. In one embodiment as shown in FIGS. 7 and 8, the resilient member 790 is a spring.

The driving engagement portion 710 of the driver 702 is formed with a third diameter d₃, and the protruding portion 712 of the driver 702 is formed with a fourth diameter d₄, and as formed, the third diameter d₃ and the fourth diameter d₄ satisfy the relationship of d₃>d₄, such that a step portion 712a is formed at the intersection of the driving engagement portion 710 and the protruding portion 712 of the driver 702.

The engagement tip 730 of the driving engagement portion 724 is formed with a shape that is complimentary to the shape of the engagement portion 748 of the anchor 742. Moreover, the first length l₁, the second length l₂, and the third length l₃ satisfy the relationship of l₁≧l₂≧l₃, or at least l₂≧l₃.

In operation, the bit 722 is received in the bore 737 of the body portion 734 of the base 732 with its engagement tip 730 engaging the engagement portion 748 of the anchor 742, the combination of the bit 722 and the base 732 is received in the bore 720 of the cap 714 through the second end 718, and the driving engagement portion 710 with the protruding portion 712 of the driver 702 is received in the bore 720 of the cap 714 through the first end 716 with a slidable engagement with the first inner surface 720a of the bore 720 of the cap 714, respectively. A fully assembled surgical instrument 700 is shown in FIG. 8(c), where it can be seen each component of the instrument is co-axial along a longitudinal axis. Thus, as the driver 702 is twisted, the driving engagement portion 710 and the protruding portion 712 of the driver 702 engage with the bit 722 and apply a pressure force to the bit 722, which in turn rotates the anchor 742 into the target area to a depth that equals the first length l₁ at which the driving engagement portion 710 of the driver 702 disengages with the first inner surface 720a of the bore 720 of the cap 714 to allow the driver 702 rotates alone. Thus, the relationship of l₂≧l₃ ensures that when the surgical instrument 700 is first placed against the skull (not shown) before driving begins, as shown in FIG. 7a, the step portion 712a of the driver 702 will be in physical contact with the first end 723 of the bit member 722. This physical contact allows the driver 702 to communicate a downward force to and through the bit member 722 and onto the second end 746b of the anchor 742. This force is required for the anchor 742 to gain initial purchase into the skull when torgue is applied.

In one embodiment, the surgical instrument 700 is configured and formed such that the second length l₂, the fourth length l₄, the fifth length l₅, and the sixth length l₆ satisfy the relationship of l₂+l₄+l₆<l₅. The relationship of l₂+l₄+l₆<l₅ ensures that, just before the base portion 746 of the anchor 742 makes contact with the skull, the torgue will be released, thereby preventing stripping. With this configuration, at the final position shown in FIG. 7c, which occurs just before the base portion 746 of the anchor 742 comes into contact with the bone, the driving engagement portion 710 of the driver 702 will be disengaged from the first inner surface 720a of the bore 720 of the cap 714, and, as a result, torque is no longer communicated to the bit member 722, which is illustrated in FIG. 7c. It is further noted that the fifth length l₅ is slightly larger than the combination l₂+l₄+l₆. In fact, in one preferred embodiment, the difference between the combination l₂+l₄+l₆ and the fifth length l₅, d=l₅−(l₂+l₄+l₆) should be small enough to assure adequate seating. In other words, the resulting final penetration distance, p=l₁−d=l₁+l₂+l₄+l₆−l₅ of the tip portion 744 of the anchor 742 into the bone, should be sufficient for the medical application.

Accordingly, the present invention provides a surgical instrument 700 for securing an anchor 742 or surgical screw in a target area of the skull of a living subject to a predetermined driving depth. As set forth above, this embodiment differs from the prior art and even the other embodiments in that it engages with the part to be driven via internal features as opposed to external features. For example the surgical instrument 700 might drive a screw using an internal crossed (Phillips), hex or slotted pattern or other geometries whereas the prior art and other embodiments might use an external hexagonal pattern or a mechanism that allows relative axial motion while transmitting rotational motion e.g. a spline. Since the part of the surgical screw that is being driven is different (inner vs outer), the internal parts of the surgical instrument 700 according to this embodiment are structure-wise significantly different from the others although the surgical instrument 700 still operates from the same basic principal of “geometry” disengagement as opposed to friction or slip disengagement to stop screw rotation. In other words, the surgical instrument 700 will properly seat the surgical screw to the proper driving depth regardless of how much or little torque the surgical screw encounters, which ensures the safety of the patient(s). This is important because the torque encountered when screwing into bone, can be different based on many physiological factors.

In sum, for the surgical instrument 700 as shown in FIGS. 7 and 8, one end of the bit member has a feature or pattern which matches a feature or pattern in the surgical screw to be driven. The upper or the other end portion of the bit is hexagonal (or other geometries) and is slidably engaged with the base member such meaning that the bit can move relative to a longitudinal axis of the base member but cannot rotate relative to the axis. The base member is mated rigidly to the cap member via screw threads or other mechanical means. The cap member in one embodiment has a hexagonal shape in the top portion, which engages with a corresponding hexagonal shape on the stem or driver. The length of the hexagonal shape on the stem or driver corresponds to the “driving depth” in FIG. 7a, or l₁, which itself may be adjustable. The Stem is slidably engaged with the Cap. The lower portion of the stem or driver is in contact with the bit member and provides downward pressure or force to the bit member. A resilient member such as a spring provides downward pressure from the bit member to the base member to ensure that the base member remains in contact with the bone or plate that the surgical screw is passing through. The resilient member also ensures that the surgical instrument 700 can operate in any position independent of gravity.

In operation, the surgical instrument 700 is rotated via twisting or rotating the stem or driver, and downward pressure is applied on and toward the surgical screw. As the surgical screw threads engage deeper into the bone in the target area, the surgical screw moves down axially relative to the top portion of the cap member (FIG. 7b). Subsequently, the stem or driver and bit member move down with the surgical screw so that progressively, less of the hexagonal interface between the stem or driver and the top portion of the cap is engaged (driving depth l₁ in FIG. 7a gets smaller). Once the driving depth has been reached (goes to zero), i.e., the engagement length has been fully traveled, the hexagonal surfaces of the stem or the driver and the cap member are no longer in contact and the stem or driver is no longer rotationally engaged with the cap member (FIG. 7c). As such the base member, which is rigidly fastened to the cap member, will now be stationary along with the bit member and surgical screw, while the stem or driver is still rotating. It is now evident to the operator of the surgical instrument 700 that the base member is stationary and that the surgical screw is properly seated.

In other words, for the surgical instrument 700, in operation, rotation is transmitted through the stem or the driver to the cap member on to the base member and then to the bit member as long as the driving depth is greater than 0. Axial force is transmitted through the stem or driver to the bit member directly.

Surgical instruments of various embodiments of the present invention can be made with different sizes or dimensions, and with proper materials such as metallic materials, plastic materials, wood materials, combinations of them, or the like.

The foregoing description of the exemplary embodiments of the invention has been presented only for the purposes of illustration and description and is not intended to be exhaustive or to limit the invention to the precise forms disclosed. Many modifications and variations are possible in light of the above teaching. For examples, in an alternative embodiment, the axial length, l₇, of the protruding portion 712 of the driver 702, can be lengthened so that the protruding portion 712 makes physical contact with the base of the hollow well 729 inside the bit member 722 beginning with the first position of the instrument, as shown in FIG. 7a, and lasting to the last position of the instrument, as shown in FIG. 7c. This physical contact is assured if the length l₇ of the protruding portion 712 is greater than or equal to the axial depth l₈ of the hollow well 729, i.e., l₇≧l8, as shown in FIG. 7a. This relationship and corresponding configuration allows the driver 702 to communicate downward force on the bit 722 by means of physical contact between the protruding portion 712 and the bottom of the hollow well 729, instead of by means of physical contact between the step portion 712a and the first end 723 of the bit member 722. As noted above, this downward force is needed at the beginning in order for the anchor to gain initial purchase into the skull when torque is applied.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

What is claimed is:

1. A surgical instrument for securing an anchor in a target area of the skull of a living subject, wherein the anchor has a tip portion and a base portion, and wherein the tip portion is configured to penetrate into the targeted area and with a first length l₁ equal to a predetermined driving depth, and the base portion configured to support the tip portion at a first end and has an engagement portion formed at an opposite, second end, comprising:
(a) a driver having a first end, an opposite, second end, and a body portion defined therebetween and along a longitudinal axis, wherein a driving engagement portion and a protruding portion protruding away from the driving engagement portion are formed at the second end along the longitudinal axis of the driver, and wherein the driving engagement portion is formed with a second length l₂ along the longitudinal axis and a shaped surface;
(b) a bit having a base portion with a first end, an opposite, second end, and a shaped surface formed therebetween, and a driving engagement portion formed with an engagement tip and protruding away from the base portion at the second end of the base portion along a longitudinal axis of the bit, wherein a bore is formed proximate to the first end and with a shape that is complimentary to the shape of the protruding portion to allow the protruding portion to be received therein;
(c) a cap having a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with a third length l₃ along the longitudinal axis and a shape that is complimentary to the shaped surface of the driving engagement portion to allow the shaped surface of the driving engagement portion to be received therein; and
(d) a base having a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein.

2. The surgical instrument of claim 1, wherein the shaped surface of the driving engagement portion is cross-sectionally hexagonal, cylindrical, oval or polygonal.

3. The surgical instrument of claim 2, wherein the shape of the first inner surface of the cap is cross-sectionally hexagonal, cylindrical, oval or polygonal.

4. The surgical instrument of claim 3, wherein the shape of the second inner surface of the cap is formed with a shape that is complimentary to the shape of the body portion of the base to allow the body portion to be received therein.

5. The surgical instrument of claim 4, further comprising fastening means for fastening the base and the cap together once the body portion of the base is received in the bore.

6. The surgical instrument of claim 3, wherein the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

7. The surgical instrument of claim 6, further comprising a resilient member configured to be received in the bore of the body portion of the base and in contact with the second end of the base portion of the bit and the step portion of the base for providing a tension force to the bit and the base, respectively.

8. The surgical instrument of claim 7, wherein the engagement tip of the driving engagement portion is formed with a shape that is complimentary to the shape of the engagement portion of the anchor.

9. The surgical instrument of claim 8, wherein the second length l₂, and the third length l₃ satisfy the relationship of l₂≧l₃.

10. The surgical instrument of claim 9, wherein in operation, the bit is received in the bore of the body portion of the base with its engagement tip engaging the engagement portion of the anchor, the combination of the bit and the base is received in the bore of the cap through the second end, and the driving engagement portion with the protruding portion of the driver is received in the bore of the cap through the first end with a slidable engagement with the first inner surface of the bore of the cap, respectively, such that as the driver is twisted, the driving engagement portion and the protruding portion of the driver engage with the bit and apply a pressure force to the bit, which in turn rotates the anchor into the target area to a desired depth at which the driving engagement portion of the driver disengages with the first inner surface of the bore of the cap to allow the driver rotates alone.

11. The surgical instrument of claim 10, wherein the resilient member comprises a spring.

12. The surgical instrument of claim 3, wherein the driving engagement portion of the driver is formed with a third diameter d₃, and the protruding portion of the driver is formed with a fourth diameter d₄, and wherein the third diameter d₃ and the fourth diameter d₄ satisfy the relationship of d₃>d₄, such that a step portion is formed at the intersection of the driving engagement portion and the protruding portion of the driver.

13. The surgical instrument of claim 1, wherein the bit is formed with a fourth length l₄ along the longitudinal axis A from the first end to the end of the driving engagement portion proximate to the engagement tip, and the base is formed with a fifth length l₅ along the longitudinal axis A from the first end to the end of the anchor engagement portion distal from the body portion, respectively.

14. The surgical instrument of claim 13, wherein the base portion of the anchor is formed with a sixth length l₆ along the longitudinal axis A, and the second length l₂, the fourth length l₄, the fifth length l₅, and the sixth length l₆ satisfy the relationship of l₂+l₄+l₆<l₅.

15. The surgical instrument of claim 1, wherein the bit member is formed with a hollow well therein.

16. The surgical instrument of claim 15, wherein the protruding portion of the driver is formed with a seventh length l₇ along the longitudinal axis A, the hollow well is formed with an axial depth or eighth length l₈ along the longitudinal axis A, and the seventh length l₇ and the eighth length l₈ satisfy the relationship of l₇>l₈.

17. A surgical instrument for securing an anchor, comprising:
(a) a driver having a first end, an opposite, second end, and a body portion defined therebetween and along a longitudinal axis, wherein a driving engagement portion and a protruding portion protruding away from the driving engagement portion are formed at the second end along the longitudinal axis of the driver, and wherein the driving engagement portion is formed with a shaped surface;
(b) a bit having a base portion with a first end, an opposite, second end, and a shaped surface formed therebetween, and a driving engagement portion formed with an engagement tip and protruding away from the base portion at the second end of the base portion along a longitudinal axis of the bit, wherein a bore is formed proximate to the first end and with a shape that is complimentary to the shape of the protruding portion to allow the protruding portion to be received therein;
(c) a cap having a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with a shape that is complimentary to the shaped surface of the driving engagement portion to allow the shaped surface of the driving engagement portion to be received therein; and
(d) a base having a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein.

18. The surgical instrument of claim 13, wherein the shaped surface of the driving engagement portion is cross-sectionally hexagonal, cylindrical, oval or polygonal.

19. The surgical instrument of claim 14, wherein the shape of the first inner surface of the cap is cross-sectionally hexagonal, cylindrical, oval or polygonal.

20. The surgical instrument of claim 15, wherein the shape of the second inner surface of the cap is formed with a shape that is complimentary to the shape of the body portion of the base to allow the body portion to be received therein.

21. The surgical instrument of claim 16, further comprising fastening means for fastening the base and the cap together once the body portion of the base is received in the bore.

22. The surgical instrument of claim 15, wherein the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

23. The surgical instrument of claim 17, further comprising a resilient member configured to be received in the bore of the body portion of the base and in contact with the second end of the base portion of the bit and the step portion of the base for providing a tension force to the bit and the base, respectively.

24. The surgical instrument of claim 18, wherein the engagement tip of the driving engagement portion is formed with a shape that is complimentary to the shape of the engagement portion of the anchor.

25. The surgical instrument of claim 20, wherein in operation, the bit is received in the bore of the body portion of the base with its engagement tip engaging the engagement portion of the anchor, the combination of the bit and the base is received in the bore of the cap through the second end, and the driving engagement portion with the protruding portion of the driver is received in the bore of the cap through the first end with a slidable engagement with the first inner surface of the bore of the cap, respectively, such that as the driver is twisted, the driving engagement portion and the protruding portion of the driver engage with the bit and apply a pressure force to the bit, which in turn rotates the anchor into the target area until the driving engagement portion of the driver disengages with the first inner surface of the bore of the cap.

26. The surgical instrument of claim 21, wherein the resilient member comprises a spring.

27. The surgical instrument of claim 14, wherein the driving engagement portion of the driver is formed with a third diameter d₃, and the protruding portion of the driver is formed with a fourth diameter d₄, and wherein the third diameter d₃ and the fourth diameter d₄ satisfy the relationship of d₃>d₄, such that a step portion is formed at the intersection of the driving engagement portion and the protruding portion of the driver.

28. A surgical instrument for securing an anchor in a target area of the skull of a living subject to a predetermined driving depth, comprising:
(a) a driver member having a first end, an opposite, second end, and a body portion defined therebetween and along a longitudinal axis, wherein a driving engagement portion is formed at the second end along the longitudinal axis of the driver, and wherein the driving engagement portion is formed with a shaped surface;
(b) a bit member having a base portion with a first end, an opposite, second end, and a shaped surface formed therebetween, and a driving engagement portion formed with an engagement tip and protruding away from the base portion at the second end of the base portion along a longitudinal axis of the bit;
(c) a cap member having a first end, an opposite, second end, and a body portion defined therebetween and forming a bore therein along a longitudinal axis, wherein inside the bore, a first inner surface is formed proximate to the first end and a second inner surface is formed between the first inner surface and the second end, respectively, wherein the first inner surface is formed with an engagement length and a shape that is complimentary to the shaped surface of the driving engagement portion to allow the driving engagement portion of the driver member to have a slidable engagement with the cap member; and
(d) a base member having a body portion with a first end, an opposite, second end, and a bore formed therebetween, and an anchor engagement portion protruding away from the second end of the body portion and with a bore in communication and co-axial with the bore of the body portion, wherein inside the bore of the body portion, a first inner surface is formed with a shape that is complimentary to the shaped surface of the bit to allow the bit to be received therein, and wherein inside the bore of the anchor engagement portion, a second inner surface is formed with a shape that is complimentary to the shape of the driving engagement portion formed with an engagement tip to allow the driving engagement portion formed with an engagement tip to slidably move therein,
wherein the engagement length of the first inner surface of the cap member equals the predetermined driving depth.

29. The surgical instrument of claim 24, wherein the shaped surface of the driving engagement portion is cross-sectionally hexagonal, cylindrical, oval or polygonal.

30. The surgical instrument of claim 25, wherein the shape of the first inner surface of the cap is cross-sectionally hexagonal, cylindrical, oval or polygonal.

31. The surgical instrument of claim 26, wherein the shape of the second inner surface of the cap is formed with a shape that is complimentary to the shape of the body portion of the base to allow the body portion to be received therein.

32. The surgical instrument of claim 27, further comprising fastening means for fastening the base and the cap together once the body portion of the base is received in the bore.

33. The surgical instrument of claim 26, wherein the first inner surface of the bore is formed with a first diameter d₁, and the second inner surface of the bore is formed with a second diameter d₂, and wherein the first diameter d₁ and the second diameter d₂ satisfy the relationship of d₁>d₂, such that a step portion is formed at the intersection of the first inner surface and the second inner surface of the bore.

34. The surgical instrument of claim 29, further comprising a resilient member configured to be received in the bore of the body portion of the base and in contact with the second end of the base portion of the bit and the step portion of the base for providing a tension force to the bit and the base, respectively.
